(19) United States
(12) Patent Application Publication
(10) Pub. No.: US 2025/0082205 A1
(43) Pub. Date: Mar. 13, 2025

(54) Method and System for Enhanced Fluorescence Tissue and Cancer Margin Imaging

(71) Applicant: CytoVeris Inc., Farmington, CT (US)

(72) Inventors: Alan Kersey, South Glastonbury, CT (US); Rishikesh Pandey, Unionville, CT (US)

(21) Appl. No.: 18/882,678

(22) Filed: Sep. 11, 2024

Related U.S. Application Data
(60) Provisional application No. 63/537,715, filed on Sep. 11, 2023.

(51) Int. Cl. A61B 5/00

(52) U.S. Cl. CPC A61B 5/0071; A61B 2505/05; A61B 2576/00

(57) ABSTRACT

A method and system for analyzing a tissue, the method includes: administering a fluorescent agent to the tissue; producing a first excitation light configured to produce an autofluorescence emission from a biomolecule present within the tissue, and a diffuse reflectance signal from the tissue; producing a second excitation light configured to produce a fluorescence emission from the fluorescent agent administered to the tissue; using a photodetector to detect the autofluorescence emissions, the diffuse reflectance signals and produce first signals representative of the autofluorescence emission and the diffuse reflectance; using the photodetector to detect the fluorescence emissions and produce second signals representative of the fluorescence emission; producing a first image using the first signals; producing a second image using the second signals; and analyzing the tissue using the first image and the second image to identify the presence of diseased tissue within the tissue.

This application claims priority to U.S. Patent Appln. No. 63/537,715 filed Sep. 11, 2023, which is hereby incorporated by reference in its entirety.

### BACKGROUND OF THE INVENTION

### 1. Technical Field

The present disclosure relates to systems and methods for in-vivo tissue analysis in general, and devices and methods for detecting diseased tissue in an intraoperative procedure in particular.

### 2. Background Information

Tumor resection surgery is often one of the first steps in the treatment for solid cancers, such as those in breast, colon, lung, pancreatic and prostate cancer, as examples. These procedures are most routinely conducted following detailed medical imaging, including X-Ray, MRI, PET, and ultrasound imaging to locate and map the tumor boundaries. The use of biopsy mapping is also taken into account. Unfortunately, these imaging modalities provide spatial resolution of the tumors to ˜ mm scale, and consequently, during the surgery do not provide the surgeon with sufficient accuracy to assure complete tumor removal based on that data.

Surgeons are skilled in using visual and tactile senses, e.g., palpation, in recognizing the attributes of cancer tissue is a macroscopic sense, but at the boundaries between tumor and normal/health tissue this can be problematic for the surgeon. At the boundaries/margins, the cancer tissue may also present a color, morphology and/or structure that resembles healthy tissue, creating a challenge for the surgeon to distinguish the tumor from the healthy tissue. Additionally, the edges of tumors tend to be diffuse and there can be regions of tumor that skip along anatomical features, for example in breast cancer, DCIS cancer can ‘skip’ along ducts and be difficult to discern via the naked eye. This limits the surgeon's ability to remove all cancer tissue during the procedure: Clinical studies have shown that up to 36% of patients have re-excisions due to positive margins and 14% of patients with negative margins after lumpectomy have cancer remaining. [1] While tumors are of many types, it is desirable for a surgeon to be able to visualize the cancer tissue and to be able to discriminate between normal and cancer tissue intraoperatively.

To address this need, advanced optical imaging approaches have been proposed for tissue analysis and cancer margin detection during cancer resection surgery. These include the use of contrast-agent-based fluorescence imaging [2, 3], diffuse reflectance imaging [4], Raman spectroscopy [5,6], hyperspectral imaging [7], optical coherence tomography [8], and autofluorescence-based imaging. [9-12]

Among the optical techniques, fluorescence offers a straightforward approach to providing interpretable and attributable diagnostic information to known biology. For example, autofluorescence signatures are generated from tissue arise due to endogenous biomolecular fluorescence signatures offer useful information that can be mapped to the functional, metabolic and morphological attributes of a biological specimen, and have therefore been utilized for diagnostic purposes. Biomolecular changes occurring in the cell and tissue state during pathological processes and disease progression result in alterations of the amount and distribution of endogenous fluorophores and form the basis for classification. Tissue autofluorescence (AF) has been proposed to detect various malignancies including cancer by measuring either differential intensity or lifetimes of the intrinsic fluorophores. Biomolecules such as tryptophan, collagen, elastin, nicotinamide adenine dinucleotide (NADH), flavin adenine dinucleotide (FAD), porphyrins, etc. present in tissue provide discernible and repeatable AF spectral patterns. While tissue AF has been proposed for cancer detection, there are three major limitations for conventional AF-based diagnosis approaches. First, traditional AF assays typically use a single excitation wavelength which obviously does not excite all the intrinsic fluorophores present in the tissue. Consequently, traditional AF does not effectively utilize the comprehensive and rich biochemical information embedded in the tissue matrix both from cells and the extracellular matrix. Second, most of the applications involving AF use a fiber probe with single-point measurement capability and are inherently slow. Third, most of the AF approaches involve simpler data analysis such as calculating redox ratio or oxygenation index ratio, and do not utilize the rich morphological information.

More recently, fluorescence-guided surgery (FGS) has been used for the detection of cancer during surgery and margin assessment. Cancer imaging using fluorescence-guided surgery typically involves the use of targeted fluorescent imaging agents, for example dyes or tracers, that are administered to the patients ahead of surgery and target cancer cells through binding to cell surface carbohydrates, free proteins, specific enzymes, or expressed cell surface receptors of the cells or those that become incorporated into the intracellular matrix through metabolic processes.

Examples of imaging agents include 1) the imaging agent Tozuleristide developed by Blaze Therapeutics, a peptide component of the molecule that has a high affinity for cancer cells. [13], 2) Pegulicianine, by Lumicell, a Cathepsin-Activatable Fluorescent Cancer Specific Probe, 3) Pafolacianine, by On-Target, a fluorescent drug that targets folate-receptors which may be overexpressed in several different cancers, [14]. In other studies, the widely used imaging agent, indocyanine green (ICG) [15], conjugated to a tumor targeting peptide has been demonstrated for breast cancer [16]. Additionally, certain prodrugs that are metabolized differentially by cancer cells can also be used as fluorescent “labels”. An example of the later includes 5-ALA, which has been used extensively for brain cancers and can also be used in other cancers for visualization, for example for the detection of positive surgical margins during radical prostatectomy in patients with prostate cancer.

One of the most important determinants of treatment success in cancer resection surgery, and overall prognosis and survivability is the ability to achieve complete tumor resection, including any residual cancer in the surgical cavity. However, these targeted/labeled approaches can show limited performance. For example in a clinical trial, the imaging agent Pegulicianine achieved a sensitivity of 49.1% specificity of 86.5%. In this trial, nearly a half of the patients (43%) had at least one false positive. This type of performance is typical of targeted agent imaging techniques due to limitations including, but not limited to: (1) non-specific agent update into healthy tissue can result in low-contrast (tumor-to-healthy) imaging, causing confounding fluorescent image interpretation; (2) the natural autofluorescence of tissue can create confounding image information; (3) tissue layers can lead to misinterpretation of fluorescence images—for example thin layers of adipose tissue over tumor can lead to different imaging results from those associated with a thin layer of fibrous or muscle tissue overlaying the tumor; and (4) The contrast/brightness of agents in the tumor tissue can lead to “blooming” effects in the imaging surrounding the agents.

These fluorescence-guided surgery methods typically use high-wavelength light, such as near-infrared (NIR) or shortwave infrared (SWIR), and therefore excite the surface tumor as well as the tumor at certain depths. Consequently, the measurement focused on measuring the cancer margin on the surface is adversely impacted by the above confounding tumor signals making precise margin delimitation difficult. Further, the intrinsic fluorescence from the cells and extracellular matrix adds a nonspecific signal to the emission signal from cancer-targeting fluorescence agents, affecting the accuracy of the margin assessment.

What is needed is an approach that can aid in the precise delineation of tumor boundaries and account for the unwanted nonspecific autofluorescence (AF) and other potentially confounding signals during tumor imaging.

### SUMMARY

According to an aspect of the present disclosure, a system for analyzing a tissue is provided. The system includes an excitation light unit, a photodetector, and a system controller. The excitation light unit is configured to selectively produce a plurality of excitation lights. Each excitation light is centered on a wavelength distinct from the centered wavelength of the other excitation lights. The plurality of excitation lights includes a plurality of first excitation lights configured to produce an autofluorescence emission from one or more biomolecules of interest present within the tissue, and a diffuse reflectance signal from the tissue. The second excitation light is configured to produce a fluorescence emission from a fluorescent agent administered to the tissue (through administration to the patient systemically, or via topical application). The photodetector is configured to detect the autofluorescence emission and the diffuse reflectance resulting from the first excitation light directed to the tissue and produce first signals representative of the autofluorescence emission and the diffuse reflectance. The photodetector is also configured to detect the fluorescence emission signal resulting from the second excitation light directed to the tissue and produce second signals representative of the fluorescence emission. The system controller is in communication with the excitation light unit, the photodetector, and a non-transitory memory storing instructions, which instructions when executed cause the system controller to: control the excitation light unit to sequentially produce the first excitation light and the second excitation light; receive and process the first signals to produce a first image representative of the first signals, and receive and process the second signals to produce a second image representative of the second signals; and analyze the tissue using the first image and the second image to identify the presence of diseased tissue within the tissue.

In any of the aspects or embodiments described above and herein, the first signals representative of the autofluorescence emission and the diffuse reflectance may be used to create a map of tissue type in the imaged tissue, and the map may be used to correct optical inaccuracies in the second signals representative of the fluorescence emission.

According to an aspect of the present disclosure, a method of analyzing a tissue is provided that includes: administering a fluorescent agent to the tissue; producing a first excitation light centered on a first wavelength, wherein the first excitation light is configured to produce an autofluorescence emission from one or more biomolecules of interest present within the tissue, and a diffuse reflectance signal from the tissue; producing a second excitation light centered on a second wavelength, wherein the second excitation light is configured to produce a fluorescence emission from the fluorescent agent administered to the tissue; using a photodetector to detect the autofluorescence emissions, the diffuse reflectance signals and produce first signals representative of the autofluorescence emission and the diffuse reflectance; using the photodetector to detect the fluorescence emissions and produce second signals representative of the fluorescence emission; producing a first image using the first signals; producing a second image using the second signals; and analyzing the tissue using the first image and the second image to identify the presence of diseased tissue within the tissue.

The foregoing features and elements may be combined in various combinations without exclusivity, unless expressly indicated otherwise. These features and elements as well as the operation thereof will become more apparent in light of the following description and the accompanying drawings. It should be understood, however, the following description and drawings are intended to be exemplary in nature and non-limiting.

### BRIEF DESCRIPTION OF THE DRAWINGS

FIG. 1 is a block diagram illustrating an embodiment of the present disclosure.

FIG. 2 is a diagrammatic illustration of a present disclosure system embodiment.

FIG. 2A is a diagrammatic illustration of a present disclosure system embodiment.

FIG. 3 is a diagrammatic illustration of a present disclosure system embodiment.

FIGS. 4A and 4B are diagrammatic images illustrating cancer margin visualization using fluorescence agents.

FIG. 5 includes examples of AF images from fluorescent agent labeled tumor tissue.

FIG. 6A is a color image of a tumor.

FIGS. 6B-6D illustrate AF images created at different excitation wavelength/emission/detection wavelength pairs.

FIG. 6E is an image of the tissue representative of an hematoxylin and eosin (H&E) stain applied to the tissue.

FIG. 6F illustrates an AF image created at different excitation wavelength/emission/detection wavelength pairs.

FIG. 6G is a diffuse reflectance image at an excitation wavelength of 618 nm.

FIGS. 7A, 7B, 8A, and 8B illustrate tissue segmentation that may be produced using tissue AF information.

FIG. 9 is a diagrammatic illustration of a probe system that may be used with the present disclosure system.

### DETAILED DESCRIPTION

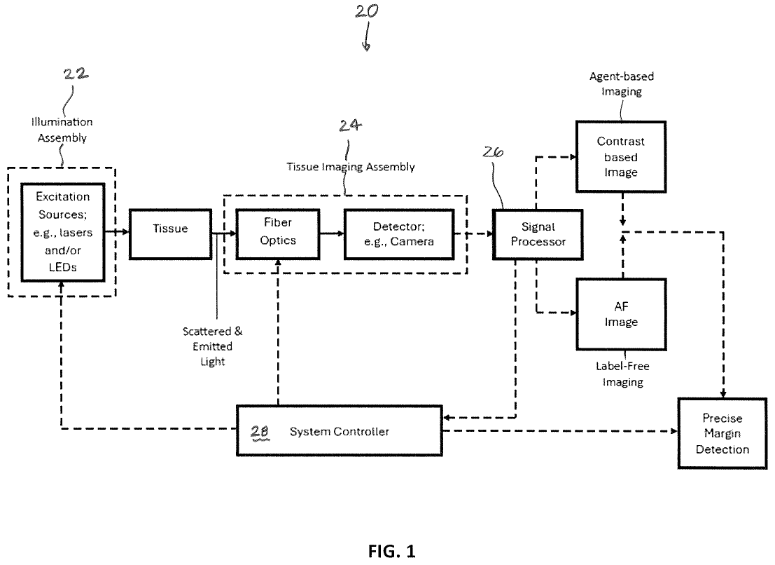

An exemplary embodiment of a present disclosure system 20 is diagrammatically illustrated in FIG. 1. The system 20 includes an illumination assembly 22 that delivers excitation wavelengths to the tissue, a tissue imaging assembly 24, a signal processor 26 that generates both agent-based and label-free tissue autofluorescence (AF) images, and a system controller 28 detailed herein. In some embodiments, the system 20 may include a display device (e.g., a monitor) configured to display tissue images and/or other relevant information. The present disclosure is not limited to the embodiment diagrammatically shown in FIG. 1.

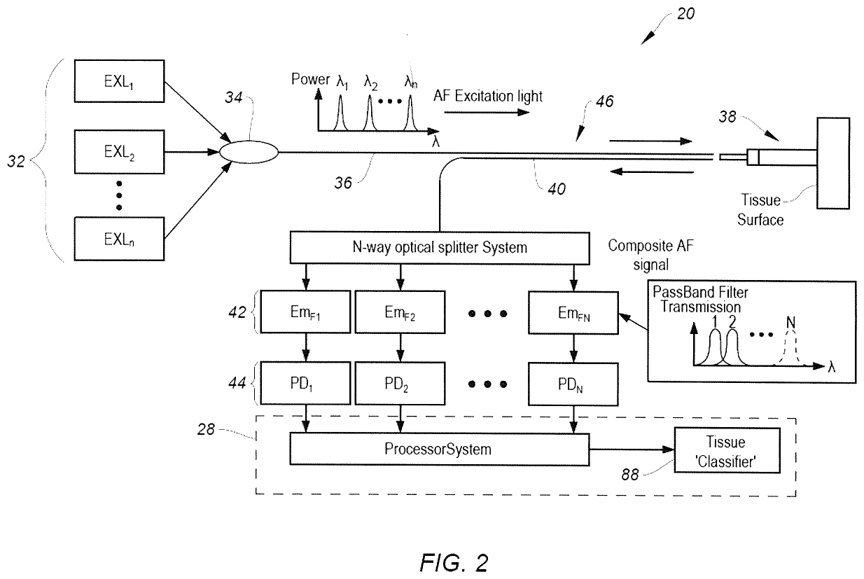

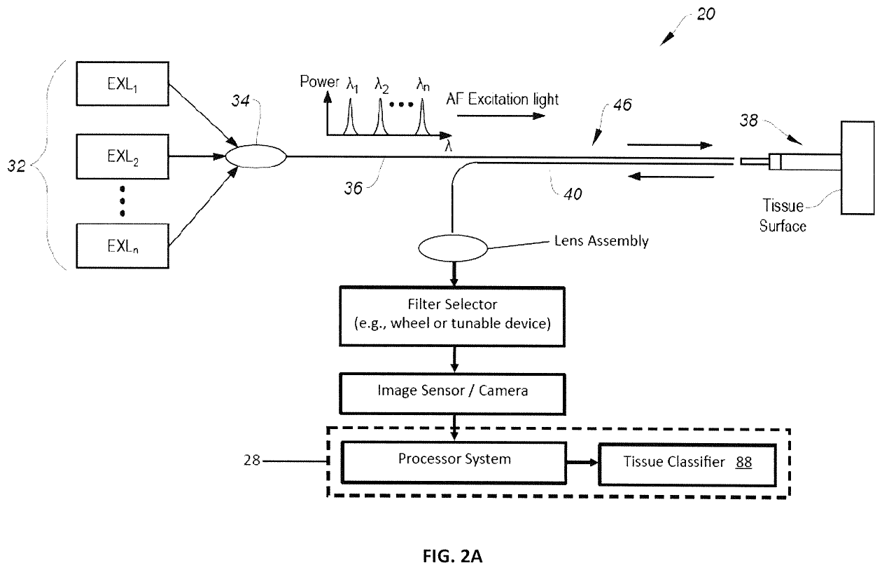

FIG. 2 diagrammatically illustrates a present disclosure system 20 embodiment that includes an excitation light unit 32, an optical switch 34, one or more first optical fibers 36, a probe 38, one or more second optical fibers 40, an emission light filter assembly 42, a photodetector arrangement 44, and a system controller 28. These system elements may be arranged as shown in FIG. 1 but are not limited to that exemplary arrangement.

The excitation light unit 32 is configured to produce excitation light centered at a plurality of different wavelengths. As will be detailed below, the term “excitation light unit” as used herein refers to a light source configured to produce excitation light that causes AF emissions and produce reflectance signals, as well as fluorescence emissions. Examples of an acceptable excitation light unit 32 include lasers and/or light emitting diodes (LEDs) each centered at a different wavelength, or a tunable excitation light source configured to selectively produce light centered at respective different wavelengths, or a source of white light (e.g., flash lamps) that may be selectively filtered to produce the aforesaid excitation light centered at respective different wavelengths. In those embodiments that include a tunable excitation light source, the tunable excitation light source may be operated to sequentially produce each of the respective excitation wavelengths. The present disclosure is not limited to any particular type of excitation light unit 32, provided the produced light can be conveyed to the tissue surface.

In the exemplary embodiment shown in FIG. 2, the excitation light unit 32 includes a plurality of independent excitation light sources (e.g., EXL₁ . . . . EXLₙ, where “n” is an integer greater than one), each operable to produce an excitation light centered at a particular wavelength and each centered on an excitation wavelength different from the others. Each of the plurality of independent excitation light sources produces excitation light centered on a different wavelength.

The respective excitation wavelengths are chosen based on either native tissue fluorophores that may be present within diseased tissue and the significance of those fluorophores relative to diseased tissue or based on the reflectance characteristics of certain tissue types and the significance of those tissue types relative to diseased tissue, or both. In other words, excitation wavelengths may be chosen that are known to produce identifiable AF emissions from native fluorophores having emission characteristics (e.g., intensity, density of signal within a given area, etc.) that provide information regarding the presence of diseased tissue (e.g., cancerous tissue) and/or to produce identifiable reflectance emissions from the tissue having characteristics that provide information regarding the presence of diseased tissue.

The excitation light unit 32 is also configured to produce excitation light at a wavelength chosen to excite an agent-based fluorescence signal. The excitation light can be optimized at the peak of the excitation efficiency of the agent used, or at any other desirable wavelength that produces adequate response to the excitation light. In some cases the agent excitation may comprise a plurality of lights at different wavelengths.

The wavelengths produced by the excitation light unit 32 are typically chosen based on the photometric properties associated with one or more biomolecules of interest. Excitation light incident to a biomolecule that acts as a fluorophore will cause the fluorophore to emit fluorescent light at a wavelength longer than the wavelength of the excitation light; i.e., via AF. Tissue may naturally include certain fluorophores such as tryptophan, collagen, elastin, nicotinamide adenine dinucleotide (NADH), flavin adenine dinucleotide (FAD), porphyrins, and the like. Biomolecular changes occurring in the cell and tissue state during pathological processes and as a result of disease progression often result in alterations of the amount and distribution of these endogenous fluorophores. Hence, diseased tissues such as cancerous tissue, due to the marked difference in cell-cycle and metabolic activity can exhibit distinct intrinsic tissue AF, or in other words an “AF signature” that is identifiable.

Embodiments of the present disclosure may utilize these AF characteristics/signatures to identify regions of diseased tissue such as cancerous tissue. Different types of diseased tissue (e.g., different types of cancerous tissue) and diseases tissue of different organs for instance breast and liver cancers may have different biomolecules/biochemicals associated therewith and the present disclosure is not therefore limited to any particular biomolecule or any particular cancer type. The AF signature can also be used to distinguish different types of health tissue, and this creates the ability to map the type and location of health tissues/structures surrounding the tumor tissue. Excitation wavelengths are also chosen that cause detectable light reflectance from tissue of interest. The detectable light reflectance is a function of light absorption of the tissue and/or light scattering associated with the tissue (this may be collectively referred to as diffuse reflectance). Certain tissue types or permutations thereof have differing and detectable light reflectance characteristics (“signatures”) at certain wavelengths. Significantly, these reflectance characteristics can provide information beyond intensity; e.g., information relating to cellular or microcellular structure such as cell nucleus and extracellular components. The morphology of a healthy tissue cell may be different from that of an abnormal or diseased tissue cell. Hence, the ability to gather cellular or microstructural morphological information (sometimes referred to as “texture”) provides another tool for determining tissue types and the state and characteristics of such tissue. The excitation light source may be configured to produce light at wavelengths in the ultraviolet (UV) region (e.g., 100-400 nm) and in some applications may include light in the visible region (e.g., 400-700 nm), and/or the near—and shortwave-infrared regions (NIR: 700-1000 nm, SWIR: 1000-2500 nm). The excitation lights are chosen based on the absorption and fluorescence characteristics of the biomolecules of interest.

The excitation light unit 32 is in direct or indirect communication with the system controller 28. In the example system 20 embodiment shown in FIG. 2, the independent excitation light sources (e.g., EXL₁ . . . . EXLₙ) are UV LEDs. The LEDs may be in communication with an LED driver that may be independent of the system controller 28 or the functionality of the LED driver may be incorporated into the system controller 28. As stated above, the present disclosure is not limited to the excitation light unit 32 example shown in FIG. 2. In some embodiments of the present disclosure, the system 20 may be configured to permit the excitation light unit 32 to switch the production of excitation light at a given wavelength on and off sequentially. In some embodiments, the present disclosure system 20 may be configured to multiplex the production of excitation light at given wavelengths.

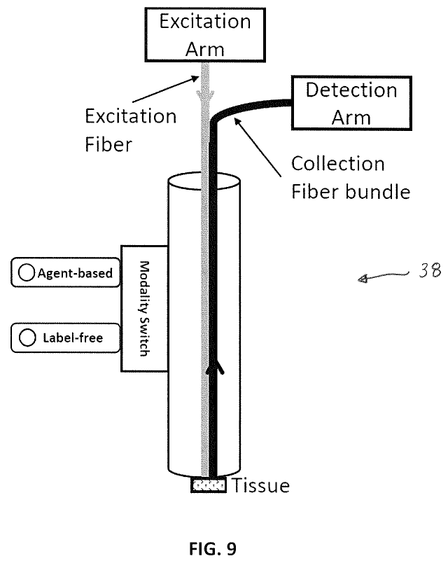

In some system 20 embodiments, the system 20 may include a probe 38 that is in communication with the excitation light unit 32 and with other components within the system 20. The probe 38 may be configured to deliver excitation light and/or capture light emitted or reflected from the tissue. An example of a probe 38 that may be used with the present disclosure system 20 is detailed in U.S. patent application Ser. No. 18/823,616, filed on Sep. 3, 2024, and commonly assigned with the present application. U.S. patent application Ser. No. 18/823,616 is hereby incorporated by reference in its entirety. FIG. 9 diagrammatically illustrates an example of a probe 38 that may be used with the present disclosure.

The system 20 embodiment example shown in FIG. 2 includes an optical switch 34. Embodiments of the present disclosure system 20 may not include an optical switch 34.

The light emitted by the tissue due to AF and that reflected from the tissue is conveyed to a photodetector; e.g., a photodetector PD₁, PD₂, . . . . PD_{N} within the photodetector arrangement 44. The light receiving conveyance structure may include a relay lens assembly, or a microscope, or the like. The present disclosure is not limited to any particular type of structure for conveying light to the photodetector.

A variety of different photodetector types (e.g., within the photodetector arrangement 44) configured to sense light emitted by the tissue due to AF and light reflected from the tissue and produce signals representative thereof may be used within the present disclosure system 20. Non-limiting examples of an acceptable photodetector include those that convert light energy into an electrical signal such as photodiodes, avalanche photodiodes, a CCD array, an ICCD, a CMOS, or the like. In general, the photodetector may take the form of a image sensor, or camera. As will be described below, the photodetector(s) are configured to detect AF emissions from the interrogated tissue and/or diffuse reflectance from the interrogated tissue and produce signals representative of the detected light and communicate the signals to the system controller 28. The system 20 embodiment example shown in FIG. 2 includes a photodetector arrangement 44 includes a plurality of photodetectors (e.g., PD₁, PD₂, . . . . PD_{N}).

The system controller 28 is in communication with system components including but not limited to the excitation light unit 32, including any LED/laser excitation drivers associate therewith, any optical filter system, and the photodetector arrangement 44. The system controller 28 may be in communication with these components to control and/or receive signals therefrom to perform the functions described herein.

The system controller 28 may include any type of computing device, computational circuit, processor(s), CPU, computer, or the like capable of executing a series of instructions that are stored in memory. The instructions may include an operating system, and/or executable software modules such as program files, system data, buffers, drivers, utilities, and the like. The executable instructions may apply to any functionality described herein to enable the system controller 28 to accomplish the same algorithmically and/or coordination of system components. The system controller 28 includes or is in communication with one or more memory devices. The present disclosure is not limited to any particular type of memory device, and the memory device may store instructions and/or data in a non-transitory manner. Examples of memory devices that may be used include read-only memory, random access memory, volatile memory, non-volatile memory, static memory, dynamic memory, flash memory, cache memory, and/or any device that stores digital information. The system controller 28 may include, or may be in communication with, an input device that enables a user to enter data and/or instructions, and may include, or be in communication with, an output device configured, for example to display information (e.g., a visual display or a printer), or to transfer data, etc. Communications between the system controller 28 and other system components may be via a hardwire connection or via a wireless connection.

Embodiments of the present disclosure may include optical filtering elements configured to filter excitation light, or optical filtering elements configured to filter emitted light (including reflected light), or both. Each optical filtering element is configured to pass a defined bandpass of wavelengths associated with an excitation light source or emitted/reflected light (e.g., fluorescence or reflectance), and may take the form of a bandpass filter. In regard to filtering excitation light, the system 20 may include an independent filtering element associated with each independent excitation light source or may include a plurality of filtering elements disposed in a movable form (e.g., a wheel or a linear array configuration) or may include a single filtering element (e.g., a tunable device) that is operable to filter excitation light at a plurality of different wavelengths or each excitation light source may be configured to include a filtering element, or the like. In regard to filtering emitted light, the system 20 may include a plurality of independent filtering elements each associated with a different bandwidth or may include a plurality of filtering elements disposed in a movable form or may include a single filtering element that is operable to filter emitted/reflected light at a plurality of different wavelengths or the like. The bandwidth of the emitted/reflected light filters are typically chosen based on the photometric properties associated with one or more biomolecules of interest. Certain biomolecules may have multiple emission or reflectance peaks. The bandwidth of the emitted/reflected light filters are typically chosen to allow only emitted/reflected light from a limited portion of the biomolecule emission/reflectance response; i.e., a portion of interest that facilitates the analysis described herein.

The system 20 embodiment example shown in FIG. 2 includes an emission light filter assembly 42 having a plurality of narrow bandpass filters (e.g., Em_{F1}, Em_{F2}, . . . . Em_{FN}, where “N” is an integer greater than one). Each narrow bandpass filter may be centered at a wavelength in the UV/visible/NIR/SWIR region, which wavelength is different from those of the other narrow bandpass filters. As stated herein, the photodetector arrangement 44 example shown in FIG. 2 includes a plurality of photodetectors (e.g., PD₁, PD₂, . . . . PD_{N}). Each photodetector may be chosen to provide optimal performance at the wavelength/spectral range of light passed by the respective narrow bandpass filter, and at low intensity levels. In some embodiments, the light intensity monitored at each photodetector can be integrated for a time duration (“T”) to increase the effective signal to noise ratio (“SNR”). Each respective photodetector produces signals representative of the filtered emitted light, and those signals are communicated to the system controller 28.

FIG. 2A diagrammatically illustrates an alternative present disclosure system 20 embodiment that includes an excitation light unit 32, an optical switch 34, one or more first optical fibers 36, a probe 38, one or more second optical fibers 40, a filter selector, an image sensor/camera, and a system controller 28. The filter selector may include a plurality of filtering elements disposed in a movable form (e.g., a wheel or a linear array configuration) or may include a single filtering element (e.g., a tunable device) that is operable to filter AF emitted/reflected light at a plurality of different wavelengths. The image sensor may be any type of device operable to convert light energy into an electrical signal for further processing.

The present application addresses the limitations of the existing art by providing a secondary image of the tissue types present in the target tissue imaging area, and allowing segmentation of that tissue. Discerning the tissue type can lead to definition of the tissue absorption and scattering characteristic at the wavelength associated with the agent-based fluorescence light, thus allowing correction of the images via the AF-based tissue segmentation map. Hence, embodiments of the present disclosure provide better/enhanced definition of the tumor boundaries. This is true for the multiple applications of fluorescence-guided surgery (FGS) in cancer surgery, e.g., whether in tumor debulking, wide local excision, whole-organ resection, and peritoneal metastases lesion identification. The present disclosure provides a method and system 20 for greatly improved tumor boundary delineation, as well as accounting for the non-specific AF signal, and requires insignificant hardware changes.

The present disclosure includes a novel dual-modal approach that enables independent and sequential acquisition of emission signals from exogenous fluorophores (agent-based) as well as from natural tissue fluorophores such as tryptophan, collagen, elastin, NADH, FAD, porphyrins, etc. In some embodiments of the present disclosure, images from the agent-based and AF measurements from the same tissue region can be co-registered and may be used to correct for tissue AF and non-specific emission signals associated with the agent-based method. The exogenous agents or dyes used in the agent-based method may be administered to the patient (e.g., intravenously), or applied topically to the target tissue, or the like. The present disclosure is not limited to any particular methodology for introducing the AF agent to the tissue of interest.

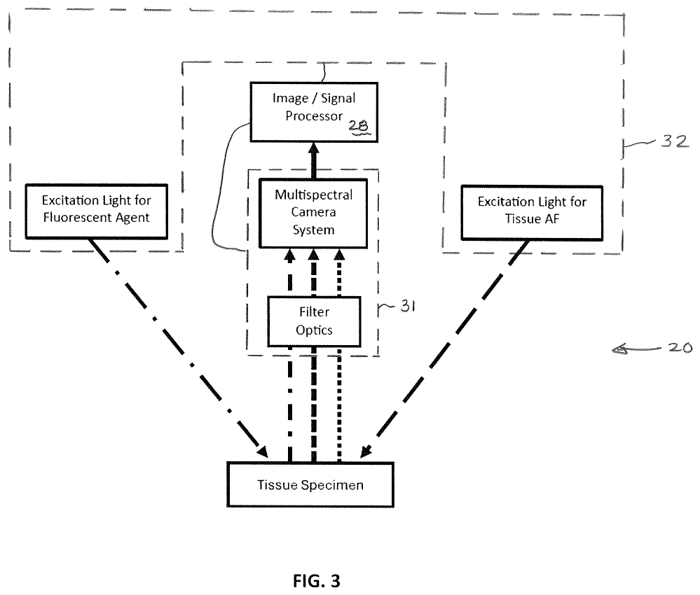

Embodiments of the present disclosure system 20, as detailed herein, include an optical imaging sub-system 31, a multi-spectral excitation light unit 32, and a system controller 28. The optical imaging sub-system 31 includes filter optics and at least one photodetector (e.g., shown as a multispectral camera system). The system 20 can operate from the UV, Visible, NIR and SWIR regions of the optical spectrum. FIG. 3 diagrammatically illustrates a present disclosure system 20 embodiment, wherein the multi-spectral excitation light unit 32 includes an excitation light source (typically configured to produce a plurality of excitation wavelengths) for the tissue AF, and an excitation light source (typically configured to produce a plurality of excitation wavelengths) for the fluorescent agent administered to the tissue.

The excitation light unit 32 is configured to selectively produce a plurality of excitation lights. Each excitation light is centered on a wavelength distinct from the centered wavelength of the other excitation lights. At least one of the excitation light centered wavelengths is configured to produce an AF emission from one or more biomolecules of interest present within the tissue, and a diffuse reflectance signal from the tissue. A second excitation light is centered at a wavelength suitable for excitation of fluorescence from an exogenous agent of the type previously described, such as 5-ALA, e.g., 405 nm. The excitation light wavelengths for the exogenous agents can be increased if multiple excitation bands are needed or if multiple agents are used.

The optical imaging sub-system may incorporate optical elements (e.g., an objective lens, a collector lens, and the like) which can comprise a surgical microscope system, or similar imaging system. The optical imaging sub-system receives light from the target tissue region under evaluation. The received light comprises optical reflectivity from the target tissue, fluorescence due to exogenous agents or dyes administered to the patient, or those applied for example topically to the target tissue, or due to tissue AF due to the natural endogenous fluorophores and/or chromophores in tissue.

The filter optics may include a controllable optical filter assembly (e.g., including a filter wheel) that receives the received light and transmits a filtered portion of the received light having a plurality of wavelengths selected by the filter. An imaging sensor (e.g., the multispectral camera system) receives the filtered light at a plurality of wavelengths from the controllable filter assembly. The imaging sensor may be monochrome image sensor, or may be configured to sense colored light at different wavelengths; e.g., an RBG camera. The imaging sensor converts the light into a plurality of electrical signals. The processor (e.g., the system controller 28) processes the electrical signals to form an image of the tissue target. In some embodiments, the processor (e.g., the system controller 28) may be configured to process the signals to create a modified image; e.g., image normalization to “reference RBG” channels.

Embodiments of the present disclosure system 20 may be configured to capture a plurality of different images. For example, an image indicative of the intensity of the fluorescent agent/label over the target tissue area. This image may be generated by illuminating the tissue at an excitation wavelength of the fluorescent agent (W_{agent-ex}), and detecting the fluorescent emission wavelength of the fluorescent agent (W_{agent-em}); i.e., light emitted at a given wavelength as a result of the fluorescent agent being excited by excitation light at a given wavelength. An image may also be generated of the multi-spectral tissue AF at each excitation wavelength (Wₑₓ₋ⱼ) associated with natural endogenous fluorophores and/or chromophores that are present within the tissue, based on the respective detection wavelengths (Wₑₘ₋ⱼ) associated with the aforesaid multi-spectral tissue AF resulting from the excitation. The subscript “ex” represents the excitation light wavelength “j” and the subscript “em” represents the tissue emission (reflectance or fluorescence) wavelength “x”. Wavelengths “j” and “x” can take values>1 and are typically 4 to 16 for multi-spectral imaging. The above represents a multispectral (also sometimes referred to as a “hyperspectral”) stack or cube of images.

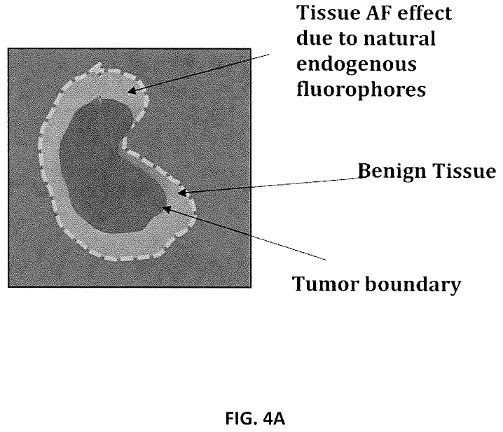

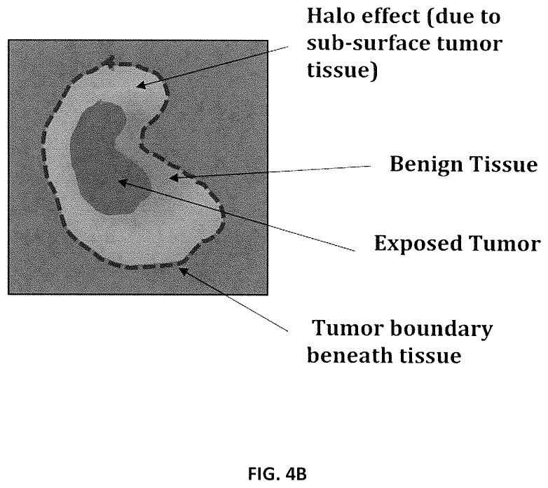

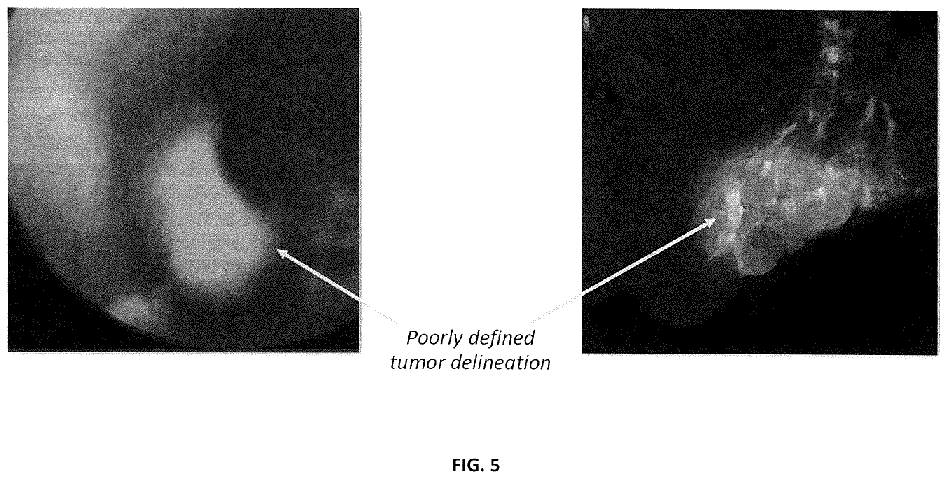

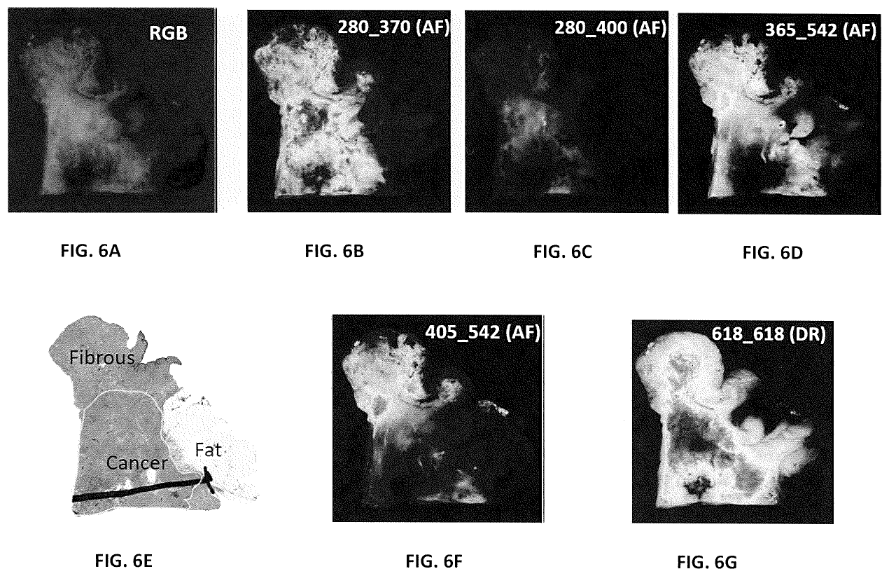

The images in FIGS. 4A and 4B illustrate cancer tumor and margin visualization using fluorescence agents, schematically illustrating the difficulty in delineating the tumor boundary. The images in FIG. 5 are examples of typical AF images from fluorescent agent labeled tumor tissue, illustrating the challenges of defining exact tumor boundaries. The images in FIG. 5 are examples of typical AF images from fluorescent agent labeled tumor tissue, illustrating the challenges of defining exact tumor boundaries. FIG. 6A is a color image of a tumor. FIGS. 6B-6D, and 6F illustrate typical AF images created at different excitation wavelength/emission/detection wavelength pairs. FIG. 6B is an image at an excitation wavelength of 280 nm and an AF emission wavelength of 370 nm. FIG. 6C is an image at an excitation wavelength of 280 nm and an AF emission wavelength of 400 nm. FIG. 6D is an image at an excitation wavelength of 365 nm and an AF emission wavelength of 542 nm. FIG. 6F is an image at an excitation wavelength of 405 nm and an AF emission wavelength of 542 nm. FIG. 6G is a diffuse reflectance image at an excitation wavelength of 618 nm. FIG. 6E is an image of the tissue representative of an hematoxylin and eosin (H&E) stain applied to the tissue. These images provide information regarding the distribution of endogenous biomolecules present within the tissue. These images can be used to train artificial intelligence/machine learning (AI/ML) algorithms to identify different tissue types, and provide tissue segmentation image or map of the target tissue area. FIGS. 2 and 2A diagrammatically illustrate the system controller including a classifier 88 that may be used in AI/ML applications. U.S. patent application Ser. No. 18/027,022, published as U.S. Patent Publication No. 2023/0366821 on Nov. 16, 2023, and U.S. patent application Ser. No. 18/567,981, published as U.S. Patent Publication No. 2024/0210321 on Jun. 27, 2024, both commonly assigned herewith and incorporated by reference in their respective entirety, disclose methodologies for utilizing artificial intelligence (including machine learning) in the analysis of tissue that may be used with the present disclosure.

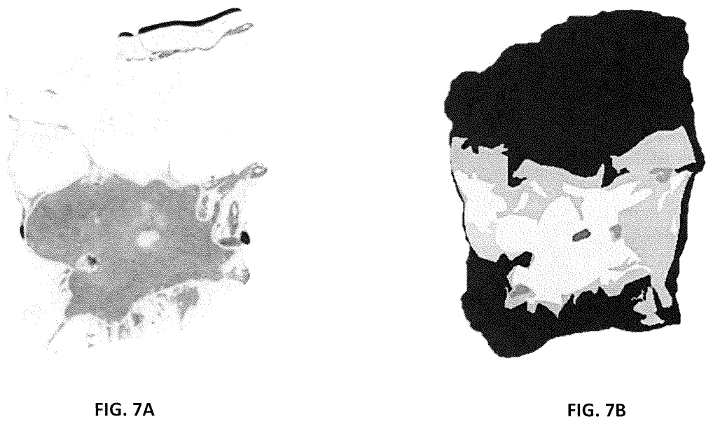

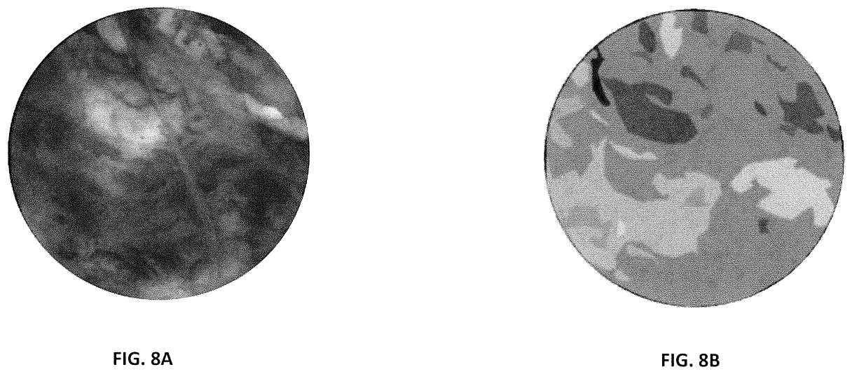

FIGS. 7A, 7B, 8A, and 8B illustrate tissue segmentation that may be produced using tissue AF information produced as described herein. Embodiments of the present disclosure may utilize a segmentation image to overlay on an agent-based fluorescence image and provide for correction by allowing the determination of, for example: (1) the natural AF light levels within the vicinity of the tumor tissue in the wavelength band associated with the agent fluorescence, which can be subtracted from the agent based image to provide increased contrast; and (2) the specific scattering and absorption characteristics of the tissue surrounding the tumor tissue, and that potentially covering or masking the tumor tissue in certain regions of the target tissue area. This “map” of optical characteristics provides the data necessary to correct the agent-based fluorescence image for optical absorption and scattering effects from sub-surface agent-based fluorescence. This enhances the definition of the agent-based fluorescence, providing better tumor boundary definition.

It should be noted that other embodiments could include a tunable laser source or sources for illumination/excitation, an optically wavelength-filtered white light source, or other means for providing a variety of excitation wavelengths. The detection imaging assembly could comprise a tunable filter, or a series of wavelength sensitive camera/imaging sensors specific to each detection wavelength of interest. Alternatively to the RBG camera, the image sensor can be a pixelated image senor that preferentially images the one or more of the plurality of wavelengths passed by the Imaging assembly

Embodiments of the present disclosure include a method for improving and enhancing fluorescence agent-based imaging. This approach comprises independent and sequential acquisition of emission signals from exogenous fluorophores (agent-based) as well as from natural tissue fluorophores using the same or different excitation sources and detection systems. The two images-one for detecting the agents and the other for natural fluorophores—are acquired from the same region of the tissue. In some cases, these images are registered and corrected for optical aberrations. The AF image is used to provide better tumor delineation as it is essentially a surface measurement. The AF image is also used to correct the nonspecific and background signal from the natural tissue fluorophores.

While the principles of the disclosure have been described above in connection with specific apparatuses and methods, it is to be clearly understood that this description is made only by way of example and not as limitation on the scope of the disclosure. Specific details are given in the above description to provide a thorough understanding of the embodiments. However, it is understood that the embodiments may be practiced without these specific details.

It is noted that the embodiments may be described as a process which is depicted as a flowchart, a flow diagram, a block diagram, etc. Although any one of these structures may describe the operations as a sequential process, many of the operations can be performed in parallel or concurrently. In addition, the order of the operations may be rearranged. A process may correspond to a method, a function, a procedure, a subroutine, a subprogram, etc.

The singular forms “a,” “an,” and “the” refer to one or more than one, unless the context clearly dictates otherwise. For example, the term “comprising a specimen” includes single or plural specimens and is considered equivalent to the phrase “comprising at least one specimen.” The term “or” refers to a single element of stated alternative elements or a combination of two or more elements unless the context clearly indicates otherwise. As used herein, “comprises” means “includes.” Thus, “comprising A or B,” means “including A or B, or A and B,” without excluding additional elements.

It is noted that various connections are set forth between elements in the present description and drawings (the contents of which are included in this disclosure by way of reference). It is noted that these connections are general and, unless specified otherwise, may be direct or indirect and that this specification is not intended to be limiting in this respect. Any reference to attached, fixed, connected or the like may include permanent, removable, temporary, partial, full and/or any other possible attachment option.

No element, component, or method step in the present disclosure is intended to be dedicated to the public regardless of whether the element, component, or method step is explicitly recited in the claims. No claim element herein is to be construed under the provisions of 35 U.S.C. 112 (f) unless the element is expressly recited using the phrase “means for.” As used herein, the terms “comprise”, “comprising”, or any other variation thereof, are intended to cover a non-exclusive inclusion, such that a process, method, article, or apparatus that comprises a list of elements does not include only those elements but may include other elements not expressly listed or inherent to such process, method, article, or apparatus.

While various inventive aspects, concepts and features of the disclosures may be described and illustrated herein as embodied in combination in the exemplary embodiments, these various aspects, concepts, and features may be used in many alternative embodiments, either individually or in various combinations and sub-combinations thereof. Unless expressly excluded herein all such combinations and sub-combinations are intended to be within the scope of the present application. Still further, while various alternative embodiments as to the various aspects, concepts, and features of the disclosures—such as alternative materials, structures, configurations, methods, devices, and components, and so on—may be described herein, such descriptions are not intended to be a complete or exhaustive list of available alternative embodiments, whether presently known or later developed. Those skilled in the art may readily adopt one or more of the inventive aspects, concepts, or features into additional embodiments and uses within the scope of the present application even if such embodiments are not expressly disclosed herein. For example, in the exemplary embodiments described above within the Detailed Description portion of the present specification, elements may be described as individual units and shown as independent of one another to facilitate the description. In alternative embodiments, such elements may be configured as combined elements. It is further noted that various method or process steps for embodiments of the present disclosure are described herein. The description may present method and/or process steps as a particular sequence. However, to the extent that the method or process does not rely on the particular order of steps set forth herein, the method or process should not be limited to the particular sequence of steps described. As one of ordinary skill in the art would appreciate, other sequences of steps may be possible.

### REFERENCES

The following references are hereby incorporated by reference in their respective entireties:
1. Dupont, et al., Ann Surg 2021; 273 (5): 876-881.
2. Q. T. Nguyen, R. Y. Tsien, Fluorescence-guided surgery with live molecular navigation-a new cutting edge. Nature Reviews Cancer 13, 653-662 (2013).
3. Q. R. Tummers et al., Real-time intraoperative detection of breast cancer using near-infrared fluorescence imaging and Methylene Blue. Eur J Surg Oncol 40, 850-858 (2014).
4. L. L. de Boer et al., Towards the use of diffuse reflectance spectroscopy for real-time in vivo detection of breast cancer during surgery. Journal of Translational Medicine 16, 367 (2018).
5. A. S. Haka et al., Margin Assessment during Partial Mastectomy Breast Surgery Using Raman Spectroscopy. Cancer Research 66, 3317 (2006).
6. W. C. Zúñiga et al., Raman Spectroscopy for Rapid Evaluation of Surgical Margins during Breast Cancer Lumpectomy. Scientific Reports 9, 14639 (2019).
7. E. Kho et al., Hyperspectral Imaging for Resection Margin Assessment during Cancer Surgery. Clinical Cancer Research 25, 3572-3580 (2019).
8. H. Schmidt et al., Evaluation of surgically excised breast tissue microstructure using wide-field optical coherence tomography. The Breast Journal 26, 917-923 (2020).
9. J. Unger et al., Real-time diagnosis and visualization of tumor margins in excised breast specimens using fluorescence lifetime imaging and machine learning. Biomed. Opt. Express 11, 1216-1230 (2020).
10. W. Zheng, W. Lau, C. Cheng, K. C. Soo, M. Olivo, Optimal excitation-emission wavelengths for autofluorescence diagnosis of bladder tumors. Int J Cancer 104, 477-481 (2003).
11. R. Pandey et al., AI-powered biomolecular-specific and label-free multi-spectral imaging rapidly detects malignant neoplasm in surgically excised breast tissue specimens “, Archives of Pathology & Laboratory Medicine, 2022.
12. T. A. Valdez et al., Multiwavelength fluorescence otoscope for video-rate chemical imaging of middle ear pathology. Anal Chem 86, 10454-10460 (2014).
13. Yamada M, et al., A first-in-human study of BLZ-100 (tozuleristide) demonstrates tolerability and safety in skin cancer patients, Contemp Clin Trials Commun. 2021 Aug. 4; 23:100830.
14. J. L. Tany, et al., A phase III study of pafolacianine injection (OTL38) for intraoperative imaging of folate receptor-positive ovarian cancer (Study 006) J Clin Oncol 41:276-284.
15. Crawford, T. et al. ICG pHLIP: A novel agent for fluorescence-guided surgery. Cancer Res 1 Jul. 2019; 79 (13_Supplement): 1956
16. M. Goto, et al., Image-guided surgery with a new tumour-targeting probe improves the identification of positive margins. eBioMedicine. 2022 February; 76:103850.
17. Stummer, W., et al. (2006). Fluorescence-guided surgery with 5-aminolevulinic acid for resection of malignant glioma: a randomised controlled multicentre phase III trial. Lancet Oncol. 7, 392-401.
18. Adam C, et al. Photodynamic diagnosis using 5-ALA for the detection of positive surgical margins during radical prostatectomy in patients with carcinoma of the prostate: a multicentre, prospective, phase 2 trial of a diagnostic procedure. Eur Urol. 2009; 55:1281-1288.
19. Multicenter clinical trial NCT03686215.

1. A system for analyzing a tissue, comprising:
an excitation light unit configured to selectively produce a plurality of excitation lights, each said excitation light centered on a wavelength distinct from the centered wavelength of the other said excitation lights, wherein the plurality of excitation lights includes a plurality of first excitation lights configured to produce an autofluorescence emission from one or more biomolecules of interest present within the tissue, and a diffuse reflectance signal from the tissue, and a second excitation light is configured to produce a fluorescence emission from a fluorescent agent administered to the tissue;
a photodetector configured to detect the autofluorescence emission and the diffuse reflectance resulting from the plurality of first excitation lights directed to the tissue and produce first signals representative of the autofluorescence emission and the diffuse reflectance, and configured to detect the fluorescence emission signal resulting from the second excitation light directed to the tissue and produce second signals representative of the fluorescence emission; and
a system controller in communication with the excitation light unit, the photodetector, and a non-transitory memory storing instructions, which instructions when executed cause the system controller to:
control the excitation light unit to sequentially produce the first excitation light and the second excitation light;
receive and process the first signals to produce a first image representative of the first signals, and receive and process the second signals to produce a second image representative of the second signals; and
analyze the tissue using the first image and the second image to identify the presence of diseased tissue within the tissue.

2. The system of claim 1, wherein the first signals representative of the autofluorescence emission and the diffuse reflectance are used to create a map of tissue type in the imaged tissue, and this map is used to correct optical inaccuracies in the second signals representative of the fluorescence emission.

3. A method of analyzing a tissue, comprising:
administering a fluorescent agent to the tissue;
producing a first excitation light centered on a first wavelength, wherein the first excitation light is configured to produce an autofluorescence emission from one or more biomolecules of interest present within the tissue, and a diffuse reflectance signal from the tissue;
producing a second excitation light centered on a second wavelength, wherein the second excitation light is configured to produce a fluorescence emission from the fluorescent agent administered to the tissue;
using a photodetector to detect the autofluorescence emissions, the diffuse reflectance signals and produce first signals representative of the autofluorescence emission and the diffuse reflectance;
using the photodetector to detect the fluorescence emissions and produce second signals representative of the fluorescence emission;
producing a first image using the first signals;
producing a second image using the second signals; and
analyzing the tissue using the first image and the second image to identify the presence of diseased tissue within the tissue.
